(19) United States
(12) Patent Application Publication
(10) Pub. No.: US 2005/0196845 A1
(43) Pub. Date: Sep. 8, 2005

(54) Method to extend enzyme activity

(76) Inventor: Yuh-Shuen Chen, Taoyuan City (TW)

Correspondence Address: TROXELL LAW OFFICE PLLC, Suite 1404, 5205 Lessburg Pike, Falls Church, VA, 22041, US

(21) Appl. No.: 10/790,042

(22) Filed: Mar. 2, 2004

(51) Int. Cl. C12P 19/04; C12N 9/24

(52) U.S. Cl. 435/101; 435/200

(57) ABSTRACT

The present invention provides a method to extend enzyme activity and stability in highly acidic environment. Enzyme extracted from natural plants, organic acids and polysaccharides are combined to form networked cross-linked polymers through cross-linkage structure, so the enzyme is stable and hard to decompose under acidic condition due to protection by networked cross-linked polymers. With increased activity and stability, the enzyme can extend its activity and residence time in alimentary canal to reach a long-term health-caring effect.

### BACKGROUND OF THE INVENTION

The biochemical and metabolic reaction in human body is very complicated. It relies on dozens of enzymes to assure the reaction process in order. Enzyme is an important matter to substance biochemical and metabolic reaction and maintaining physiological function. It exists in natural plants and living species to a great extent. Enzyme has protein structure. It has functions to inhibit and kill bacteria, decompose wastes, eliminate drug hazards and public hazardous materials. Our body has weakened or damaged cells everyday. We need to enhance the enzymes produced by our body or take fresh enzymes to satisfy the needs of cells.

The nutritions taken by human body do not decompose, form cells and synthesize necessary nutritions by themselves. They rely on enzymes to promote metabolism. There are natural enzymes in cells to complete biochemical function for human tissues and organs. For human body, it not only needs to synthesize enzymes for itself, but also needs to take active enzymes from natural foods. So human body can maintain normal metabolism and biochemical function. Health is also enhanced in this way.

Most foods we eat are processed. The enzymes contained in foods change their structure and protein characteristics under acidic and basic environment in human alimentary canal. Consequently, the enzyme activity is damaged and the enzyme loses its original function.

### SUMMARY OF THE INVENTION

Based on the need of active enzymes by human, the inventor provides a method to extend enzyme activity to solve the drawback that enzyme activity is damaged under highly acidic environment in human alimentary canal.

The present invention provides a method to extend enzyme activity and stability in highly acidic environment. Enzyme extracted from natural plants, organic acids and polysaccharides are combined to form networked cross-linked polymers through cross-linkage structure, so the enzyme is stable and hard to decompose under acidic condition due to protection by networked cross-linked polymers. With increased activity and stability, the enzyme can extend its activity and residence time in alimentary canal to reach a long-term health-caring effect.

### BRIEF DESCRIPTION OF THE DRAWINGS

FIG. 1 is an illustration for enzyme molecules covered through cross-linking reaction in the present invention.

FIG. 2 is the comparison of residual enzyme activity between the produced enzyme by the method in the invention and the general enzyme under a highly acidic condition.

FIG. 3 is the comparison of residual enzyme activity between the produced enzyme by the method in the invention and the general enzyme under a highly acidic condition of pH=3.0.

### DETAILED DESCRIPTION OF THE PREFERRED EMBODIMENT

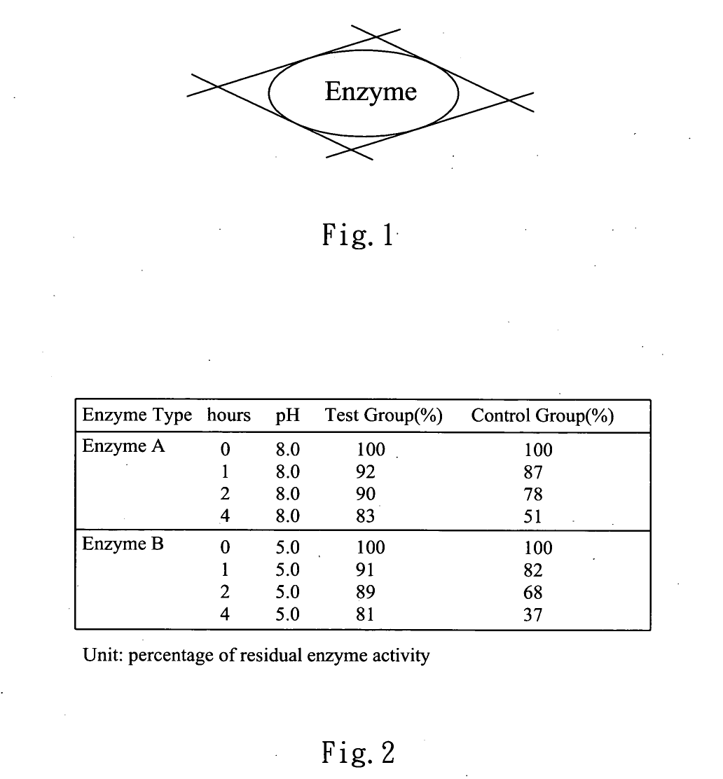

The method to extend enzyme activity in the present invention is to conduct cross-linking reaction on enzymes extracted from natural plants, organic acids and polysaccharides by hitting them with electron beam. The networked cross-linked polymers produced in the process protect the enzymes by covering the enzymes inside the polymers (as shown in FIG. 1), so the enzymes are hard to decompose under highly acidic condition. Due to increased activity and stability, the enzymes in human alimentary canal have extended activity and residence time and can provide a long-term health-caring effect.

The above-mentioned organic acids include Latic acid, Malic acid or Tartaric acid. The polysaccharides include 1,4 (−2-amino-2-deoxy-β-D-glucan), Acidified starch or Polygluco-mannose. The organic acids, the polysaccharides and the enzymes undergo 15˜25 kgy electron beam treatment for 10˜30 minutes. Then the molecules are cross-linked to cover the enzymes inside networked cross-linked polymers.

The following is to perform a comparison test of activity change under highly acidic condition for the enzyme produced by the invention (the test group) and the general enzyme (the control group). For test results, refer to FIG. 2 with enzyme activity comparison table. From the table, it is known that under a basic condition pH=8.0 for continuous one hour, the test group and the control group have the residual enzyme activity 92% and 87%, respectively. The change is minimal and no significant difference exists between the two groups. Under a basic condition pH=8.0 for continuous two hours, the test group has a residual enzyme activity 90%, while the control group has a residual activity 75%. We start seeing more change in residual enzyme activity. Under a basic condition pH=8.0 for continuous four hours, the test group has a residual enzyme activity 83%, while the control group has a residual activity 51%. The difference between the two groups becomes larger. Further, under an acidic condition pH=5.0, the residual enzyme activity difference between the two groups becomes significantly greater.

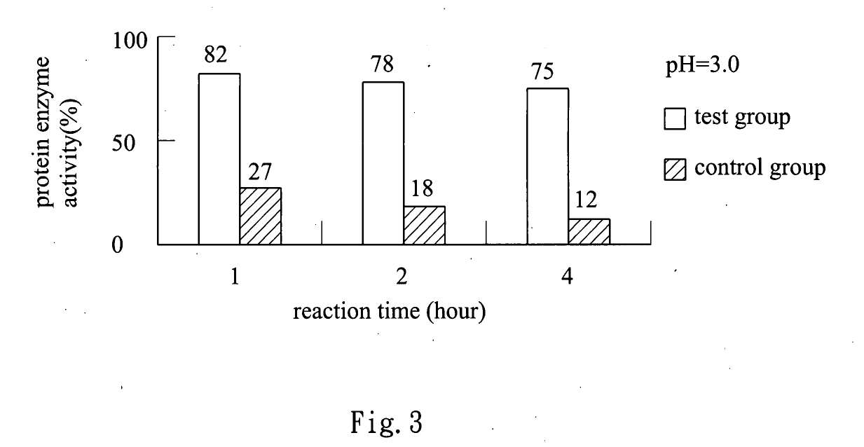

As shown in FIG. 3, under an acidic condition pH=3.0 for continuous one hour, the test group has a residual enzyme activity 82%, while the control group has a residual activity 27%. Under the same condition as above for continuous two hours, the test group has a residual enzyme activity 78%, while the control group has a residual activity 18%. Under the same condition as above for continuous four hours, the test group has a residual enzyme activity 75%, while the control group has a residual activity 12%. Thus, a dramatic difference is found.

The above comparison test indicates the cross-linking method in the present invention can cover the enzymes inside the cross-linked polymers derived through cross-linkage of enzymes, organic acids and polysaccharides. Under the protection by the cross-linked polymers, the enzymes can maintain activity for more than 4 hours under highly acid condition in human alimentary canal. The benefits of extending enzyme activity and residence time achieve a long-term health-caring effect and prove a commercial value.

1. A method to extend enzyme activity is to conduct cross-linking reaction on enzymes extracted from natural plants, organic acids and polysaccharides by hitting them with electron beam. The networked cross-linked polymers produced in the process protect the enzymes by covering the enzymes inside the polymers, so the enzymes are hard to decompose under highly acidic condition. Due to increased activity and stability, the enzymes in human alimentary canal have extended activity and residence time.

2. As described in claim 1 for a method to extend the enzyme activity, the organic acids include Latic acid, Malic acid or Tartaric acid.

3. As described in claim 1 for a method to extend the enzyme activity, the polysaccharides include 1,4 (−2-amino-2-deoxy-β-D-glucan), Acidify starch or Polygluco-mannose.

4. As described in claim 1 for a method to extend the enzyme activity, the organic acids, the polysaccharides and the enzymes undergo 15˜25 kgy electron beam treatment for 10˜30 minutes. Then the molecules are cross-linked to cover the enzymes inside networked cross-linked polymers.
